(19) United States
(12) Patent Application Publication
(10) Pub. No.: US 2025/0037863 A1
(43) Pub. Date: Jan. 30, 2025

(54) INFORMATION PROCESSING METHOD

(71) Applicant: NEC Corporation, Minato-ku,Tokyo (JP)

(72) Inventors: Tasuku KITADE, Tokyo (JP); Masanori TSUJIKAWA, Tokyo (JP)

(73) Assignee: NEC Corporation, Minato-ku, Tokyo (JP)

(21) Appl. No.: 18/714,165

(22) PCT Filed: Dec. 10, 2021
(86) PCT No.: PCT/JP2021/045654

§ 371 Date: May 29, 2024

(51) Int. Cl. G16H 50/20

(52) U.S. Cl. CPC G16H 50/20

(57) ABSTRACT

An information processing device 100 of the present invention includes: a transforming unit 121 that maps correct answer data included in learning data onto a nonlinear space, the learning data including state data representing a state of a predetermined person, and the correct answer data representing the physical condition of the predetermined person at the time of acquisition of the state data; and a generating unit 122 that generates an estimation model to be used for estimating the physical condition of a target person by learning using the state data as an explanatory variable, and the mapped correct answer data as a response variable.

### TECHNICAL FIELD

The present invention relates to an information processing method, an information processing device, and a program for estimating the physical condition of a person.

### BACKGROUND ART

As a method of estimating stress on a person, there is a known method in which a model for calculating a stress value is generated by learning in advance, and the stress value is calculated by inputting, to the model, causal data for estimating stress such as biometric data from the person. At this time, the model is generated by learning using, as an explanatory variable, feature values of biometric data about people, and using, as a response variable, stress values of the people obtained in a questionnaire or the like. For example, a stress estimation method using a model is described in Patent Literature 1.

### CITATION LIST

### Patent Literature

Patent Literature 1: WO 2021/090402

### SUMMARY OF INVENTION

### Technical Problem

However, there is a fear that, in the method in which a model is generated as mentioned above, much of learning data is data about typical people, that is, people who are not highly stressed or who are not stressed little, and data with stress values that are extremely concentrated around the median is collected. Therefore, in a case where a model is generated by learning using data that is concentrated around the median, there is a problem that the precision of stress estimation of people who are highly stressed or who are stressed little deteriorates. As a result, it is difficult to estimate stress more precisely. In addition, what is difficult to estimate is not limited to stress, but it is similarly difficult to highly precisely estimate the physical condition of a person like physical and mental fatigue, or inner condition.

Therefore, an object of the present invention is to provide an information processing method that can solve the problem mentioned above that it is difficult to estimate physical condition more precisely.

### Solution to Problem

An information processing method according to one aspect of the present invention includes:
mapping correct answer data included in learning data onto a nonlinear space, the learning data including state data representing a state of a predetermined person, and the correct answer data representing physical condition of the predetermined person at time of acquisition of the state data; and
generating an estimation model to be used for estimating physical condition of a target person by learning using the state data as an explanatory variable, and the mapped correct answer data as a response variable.

In addition, an information processing device according to one aspect of the present invention includes:
a transforming unit that maps correct answer data included in learning data onto a nonlinear space, the learning data including state data representing a state of a predetermined person, and the correct answer data representing physical condition of the predetermined person at time of acquisition of the state data; and
a generating unit that generates an estimation model to be used for estimating physical condition of a target person by learning using the state data as an explanatory variable, and the mapped correct answer data as a response variable.

In addition, a program according to one aspect of the present invention causes an information processing device to execute processes of:
mapping correct answer data included in learning data onto a nonlinear space, the learning data including state data representing a state of a predetermined person, and the correct answer data representing physical condition of the predetermined person at time of acquisition of the state data; and
generating an estimation model to be used for estimating physical condition of a target person by learning using the state data as an explanatory variable, and the mapped correct answer data as a response variable.

### ADVANTAGEOUS EFFECTS OF INVENTION

By being configured in the manners described above, the present invention makes it possible to estimate physical condition more precisely.

### BRIEF DESCRIPTION OF DRAWINGS

FIG. 1 is a block diagram depicting the configuration of a stress estimating device in a first exemplary embodiment of the present invention.

FIG. 2 is a figure depicting an example of a test for acquiring stress-related subjective data about a person input to the stress estimating device disclosed in FIG. 1.

FIG. 3 is a figure depicting examples of functions to be used when a model is generated at the stress estimating device disclosed in FIG. 1.

FIG. 4 is a flowchart depicting an operation performed by the stress estimating device disclosed in FIG. 1.

FIG. 5 is a flowchart depicting an operation performed by the stress estimating device disclosed in FIG. 1.

FIG. 6 is a block diagram depicting the hardware configuration of an information processing device in a second exemplary embodiment of the present invention.

FIG. 7 is a block diagram depicting the configuration of the information processing device in the second exemplary embodiment of the present invention.

FIG. 8 is a flowchart depicting an operation performed by the information processing device in the second exemplary embodiment of the present invention.

### DESCRIPTION OF EMBODIMENTS

### First Exemplary Embodiment

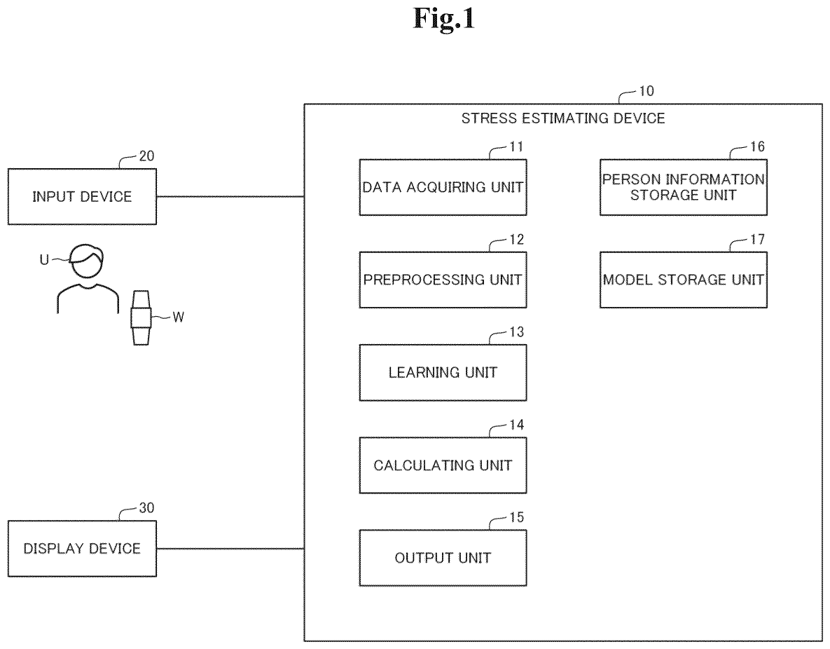

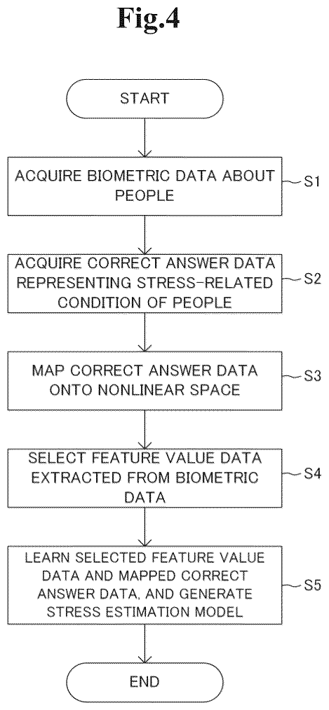

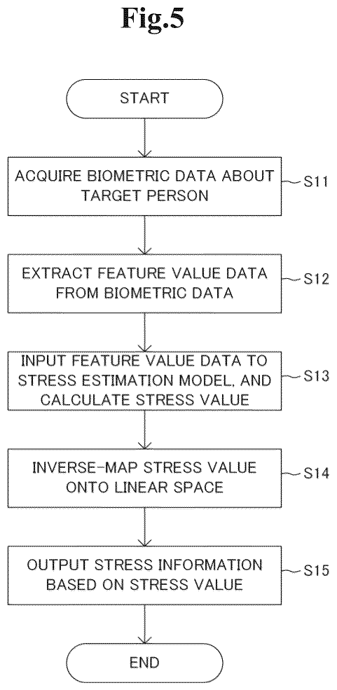

A first exemplary embodiment of the present invention is explained with reference to FIG. 1 to FIG. 5. FIG. 1 to FIG. 3 are figures for explaining the configuration of a stress estimating device, and FIG. 4 to FIG. 5 are figures for explaining processing operations performed by the stress estimating device.

### Configuration

A stress estimating device 10 (information processing device) in the present invention is used for estimating stress on a person. For example, the stress estimating device 10 is used for calculating a stress value in a predetermined situation where a person is doing her/his duties or the like at the workplace where she/he belongs. It should be noted that the stress estimating device 10 in the present invention may calculate stress on a person in any situation. In addition, the present invention can be applied not only to estimation of stress, but also to estimation of the physical condition of a person like physical and mental fatigue, or inner condition. That is, a stress value mentioned in the present embodiment is an example of a value representing the physical condition of an estimation-target person, and, as other examples of the value representing the physical condition, the value may be any value such as a fatigue degree representing the degree of fatigue, or some index value representing condition.

The stress estimating device 10 is configured using one or more information processing devices including an arithmetic device and a storage device. Then, as depicted in FIG. 1, the stress estimating device 10 includes a data acquiring unit 11, a preprocessing unit 12, a learning unit 13, a calculating unit 14, and an output unit 15. Respective functions of the data acquiring unit 11, the preprocessing unit 12, the learning unit 13, the calculating unit 14, and the output unit 15 can be realized by the arithmetic device executing programs that are stored on the storage device, and are for realizing the respective functions. In addition, the stress estimating device 10 includes a person information storage unit 16 and a model storage unit 17. The person information storage unit 16 and the model storage unit 17 are configured using the storage device. Hereinbelow, the respective configurations are mentioned in detail.

The data acquiring unit 11 acquires data to be used for estimating stress on a person. First, the data acquiring unit 11 acquires learning data for generating, by machine learning, a stress estimation model to be used for calculating a stress value. The learning data is data about certain many people (predetermined people), and is stored on the person information storage unit 16 as mentioned later.

Specifically, as learning data, the data acquiring unit 11 acquires state data representing states of people in predetermined situations such as situations where the people are doing their duties at their workplaces or the like. For example, the state data is biometric data which is various information generated from the bodies of the people. For example, the biometric data is heart rates, eye opening degrees, and the like. For example, as the state data, the data acquiring unit 11 acquires biometric data such as a heart rate of a person U via a measurement device such as a wearable terminal W worn by a person U as depicted in FIG. 1, or an eye opening degree extracted from a facial image of the person U captured with a camera which is not depicted. At this time, the data acquiring unit 11 obtains biometric data by measurement while making distinctions between people and between periods, and acquires the biometric data as the state data. It should be noted that the data acquiring unit 11 may acquire any biometric data as the state data about the people using any measurement device. The data acquiring unit 11 not necessarily acquires biometric data, but may acquire other data representing states of the people.

In addition, as learning data, the data acquiring unit 11 acquires correct answer data representing the stress-related condition of the predetermined people at the time of acquisition of the biometric data from the people as mentioned above. For example, the correct answer data is data based on stress-related subjective data about the people. Therefore, during duties, at the end of duties, or when a predetermined period has passed after duties, the data acquiring unit 11 asks the people preset questions via an input device 20, acquires and totals answers from the people U, and thereby acquires, as the correct answer data, data based on stress-related subjective data about the people U. At this time, the data acquiring unit 11 acquires the correct answer data while making distinctions between people and periods.

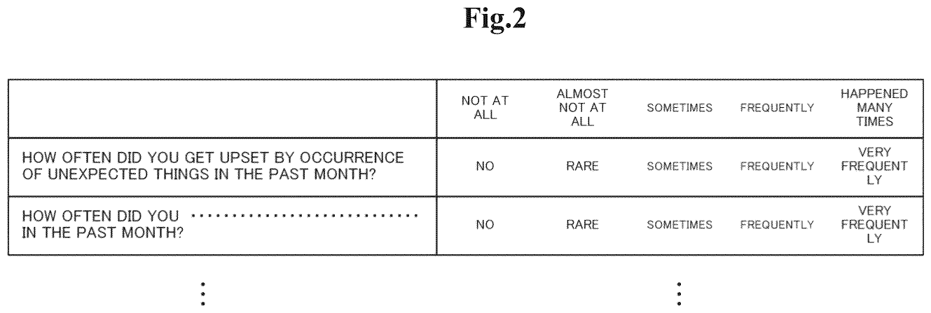

As an example, as the correct answer data which is data based on stress-related subjective data about the people, the data acquiring unit 11 acquires “PSS scores” obtained by totaling “perceived stress scales (Perceived Stress Scales (PSSs)).” Here, PSSs include 14 preset question items for asking a user about how she/he is feeling about things happening to her/him as depicted in FIG. 2 as an example, and five levels of answers are prepared. The five levels of answers are given scores from 0 to 4, and the total of the scores of answers to 10 computation-target question items in the 14 questions is calculated as a PSS score. Therefore, the value of the PSS score ranges from 0 to 40. As an example, the data acquiring unit 11 causes the input device 20 like an information processing terminal operated by a person U as depicted in FIG. 1 to display PSS questions like the ones depicted in FIG. 2, acquires answers input by the person U to the input device 20, and acquires a PSS score by totaling the answers. It should be noted that the data acquiring unit 11 does not necessarily acquire, as the correct answer data, data based on stress-related subjective data about the people, but may acquire, as the correct answer data, data representing stress-related condition acquired by other methods.

Then, the data acquiring unit 11 stores, in advance as the learning data and on the person information storage unit 16, the state data and the correct answer data acquired from the predetermined people as mentioned above in association with each person and each period. For example, in a case where the data acquiring unit 11 acquires the state data and the correct answer data from the people every month, the data acquiring unit 11 stores monthly state data and correct answer data about each person in association with each other.

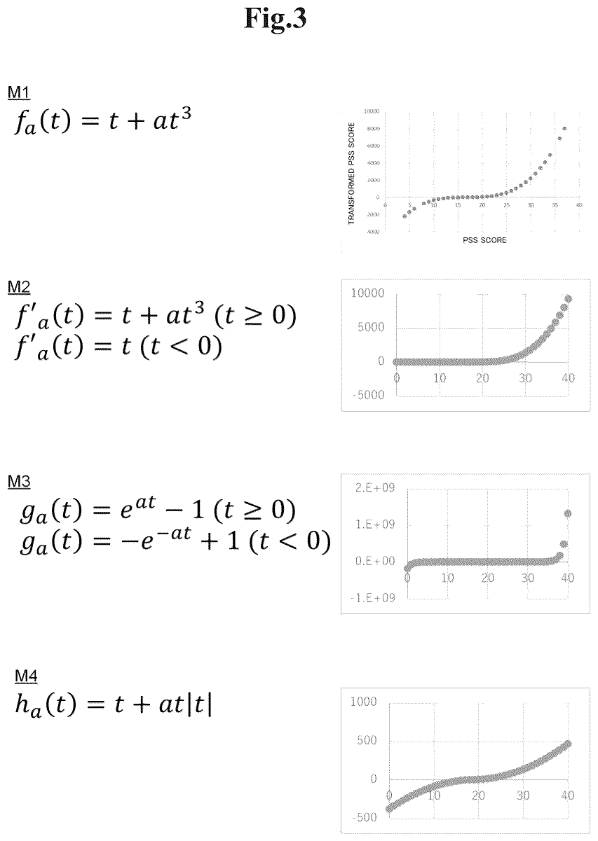

Before machine learning is performed using the learning data including the state data and correct answer data acquired and stored on the person information storage unit 16 as mentioned above, the preprocessing unit 12 (transforming unit) performs preprocessing on the learning data. First, the preprocessing unit 12 maps, onto a nonlinear space, correct answer data in a dataset including state data and the correct answer data. At this time, the preprocessing unit 12 desirably maps, onto the nonlinear space, the correct answer data using a monotonically increasing function having a rate of change which is equal to or greater than 1 such that the magnitude relationship of the correct answer data does not change before and after the mapping. Furthermore, the preprocessing unit 12 desirably maps, onto the nonlinear space, the correct answer data using a function for which an inverse function can be defined, such that a stress value calculated using a model can be inverse-map onto a linear space as mentioned later. Therefore, it is assumed in the present embodiment that the correct answer data is mapped onto the nonlinear space using any of cubic functions like the ones represented by reference signs M1 and M2 in FIG. 3, an exponential function like the one represented by a reference sign M3 in FIG. 3, and a quadratic function like the one represented by a reference sign M4 in FIG. 3. Note that it is assumed that “t” included in each function is “x-med,” “x” represents a PSS score which is the correct answer data, and “med” is the median of the overall distribution of the correct answer data. By mapping the correct answer data onto the nonlinear space using each function like the ones depicted in FIG. 3 in this manner, the weight of a value close to the median can be increased, and the weight of a value far from the median can be increased even in a case where the correct answer data is concentrated around the median as represented by graphs on the right side in FIG. 3. Note that whereas it is desirable if the quadratic function that can make weights uniform in the functions depicted in FIG. 3 is used, any function may be used, and any function not depicted in FIG. 3 may be used.

Next, the preprocessing unit 12 extracts a plurality of types of feature value from biometric data which is state data in learning data about each person and each period, and generates feature value data. For example, from biometric data acquired in a time series, the preprocessing unit 12 extracts a plurality of types of feature value data such as the average, variance/standard deviation, maximum value, minimum value, or quartile in the time domain, the average, variance, or quartile in the frequency domain, or furthermore the power spectrum peak of a histogram. Then, the preprocessing unit 12 associates mapped correct answer data with each type of feature value data, and generates a dataset of the feature value data and the mapped correct answer data. That is, the preprocessing unit 12 generates a dataset of feature value data and mapped correct answer data about each person in each period. As an example, in a case where learning data including state data and mapped correct answer data of three months has been acquired from one person, and five types of feature value data have been extracted from the state data, five patterns of datasets are generated from data of each month, and fifteen patterns of datasets are generated from the data of the three months. Then, such datasets are generated for each person. Note that the preprocessing unit 12 may generate a plurality of types of feature value data from the state data before the correct answer data is mapped onto the nonlinear space as mentioned above. In this case, the preprocessing unit 12 associates correct answer data with each type of feature value data, generates datasets of feature value data and correct answer data, thereafter maps correct answer data in each dataset onto the nonlinear space, and generates datasets of feature value data and mapped correct answer data.

Next, the preprocessing unit 12 selects types of feature value data to be used for machine learning as learning data from feature value data extracted from the state data as mentioned above. At this time, the preprocessing unit 12 calculates a correlation value representing a degree of correlation between each type of feature value data and mapped correct answer data, and selects types of feature value data on the basis of the correlation values. For example, regarding mapped correct answer data and feature value data included in all datasets belonging to a type, the preprocessing unit 12 calculates a correlation value representing a degree to which the data distribution of the mapped correct answer data and the data distribution of the feature value data are approximated to or correlated with each other in accordance with a preset criterion. Then, a correlation value is calculated for each type. That is, in a case where there are five types of feature value data mentioned above, a correlation value of each of the five types is calculated, and the top N types of feature value data having the highest correlation values, or types of feature value data having correlation values exceeding a preset threshold are selected. Note that the preprocessing unit 12 may select types of feature value data in accordance with any criterion. For example, in a case where, before correct answer data is mapped, feature value data is extracted from state data, and datasets of feature value data and correct answer data are generated, the preprocessing unit 12 may select feature value data on the basis of the distributions of the correct answer data and the feature value data before mapping, and thereafter map, onto the nonlinear space, correct answer data similarly to the manner mentioned above.

The learning unit 13 (generating unit) performs machine learning using, as learning data, datasets of feature value data selected as mentioned above, and correct answer data mapped onto the nonlinear space, and generates a stress estimation model (estimation model). Specifically, the learning unit 13 performs the machine learning using, as learning data, all datasets including the selected types of feature value data, using the feature value data as an explanatory variable, and using the mapped correct answer data as a response variable. Thereby, the generated stress estimation model can be a model configured to calculate, as a value mapped onto the nonlinear space, a value from 0 to 40 similarly to a PSS score mentioned above using biometric data about a person U as an input. Then, the learning unit 13 stores, on the model storage unit 17, the stress estimation model generated by the machine learning.

Next, functions of respective units at the time of estimation of stress on a target person (target person) using the stress estimation model after the stress estimation model is generated as mentioned above are explained. For example, it is assumed, as a scene, that the target person U belongs to a workplace, and stress on the target person U when the target person U is in a predetermined situation such as a situation where she/he is doing her/his duties at the workplace is estimated.

The data acquiring unit 11 acquires state data (target person measurement data) to be used for estimating stress on the target person U. Specifically, the data acquiring unit 11 obtains, by measurement, and acquires state data which is biometric data from the person U in a case where she/he is in a situation such as a situation where she/he is doing her/his duties at the workplace which is a scene where stress on the target person U is actually estimated. For example, the data acquiring unit 11 acquires, as the biometric data, a heart rate of the person U via a measurement device such as the wearable terminal W worn by the person U similarly to the manner mentioned above, or acquires, as the biometric data, an eye opening degree extracted from a facial image of the person U captured with a camera which is not depicted. At this time, the data acquiring unit 11 acquires the biometric data at preset timings, for example at certain time intervals, from the person U in a situation such as a situation where she/he is doing her/his duties at the workplace.

Every time biometric data is acquired from the person U as mentioned above, the calculating unit 14 calculates feature value data of the biometric data. At this time, the calculating unit 14 extracts, from the biometric data, feature value data (target person feature value data) of the same types as types of feature value data selected at the preprocessing unit 12 as mentioned above. Note that the calculating unit 14 may calculate all preset types of feature value data from the biometric data, and extract feature value data of the same types as the selected types from the feature value data.

Then, the calculating unit 14 reads out the stress estimation model stored on the model storage unit 17, and, by inputting the feature value data of the same types as the feature value data selected at the preprocessing unit 12 to the stress estimation model, calculates a stress value which is an output therefrom. That is, after the person U has started her/his duties, the calculating unit 14 calculates the stress value at preset timings such as at certain time intervals at which biometric data is acquired from the person U as mentioned above. Note that, for example, the timings at which the stress value is calculated may be during duties, and the stress value may be calculated at any intervals such as every month.

Furthermore, the calculating unit 14 (inverse transforming unit) inverse-maps, onto the linear space, the stress value calculated using the stress estimation model as mentioned above. At this time, the calculating unit 14 inverse-maps, onto the linear space, the stress value calculated using the stress estimation model using an inverse function of a function used when correct answer data is mapped onto the nonlinear space before learning at the preprocessing unit 12.

The output unit 15 outputs information based on the stress value calculated at the calculating unit 14 and inverse-mapped onto the linear space as mentioned above. For example, every time the stress value is calculated, in a case where the stress value exceeds a preset criterion value, on the basis of which it is determined whether stress is high, the output unit 15 outputs an instruction to cause a display device 30 of an information processing device operated by an administrator at the workplace administering the person U to display information to that effect (alert). Alternatively, every time the stress value is calculated, the output unit 15 may always output an instruction such that the stress value itself, that is, time-series changes of the stress value of the person U, is displayed, or may output any data based on the stress value. In addition, the output unit 15 may output data based on the stress value to any person such as the target person U.

### Operation

Next, operations performed by the stress estimating device 10 mentioned above are explained mainly with reference to flowcharts in FIG. 4 to FIG. 5. First, an operation to be performed when the stress estimation model is generated by machine learning is explained with reference to the flowchart in FIG. 4.

The stress estimating device 10 acquires learning data for generating, by machine learning, a stress calculation model to be used for calculating a stress value. Specifically, as learning data, the stress estimating device 10 acquires state data representing states of people in predetermined situations such as situations where the people are doing their duties at their workplaces or the like. For example, as the state data, the stress estimating device 10 acquires biometric data such as a heart rate of a person U via a measurement device such as the wearable terminal W worn by a person U (step S1).

Next, as learning data, the stress estimating device 10 acquires correct answer data representing the stress-related condition of the predetermined people at the time of acquisition of the biometric data from the people as mentioned above (step S2). For example, the correct answer data is data based on stress-related subjective data about the people, and, as an example, “PSS scores” obtained by totaling “perceived stress scales (Perceived Stress Scales (PSSs))” are acquired.

Next, before machine learning is performed using the learning data including the state data and the correct answer data acquired as mentioned above, the stress estimating device 10 performs preprocessing on the learning data. First, as the preprocessing, the stress estimating device 10 maps, onto a nonlinear space, the correct answer data included in a dataset including state data and correct answer data (step S3). At this time, for example as depicted in FIG. 3, the stress estimating device 10 maps, onto the nonlinear space, the correct answer data using a function for which an inverse function can be defined and which is a monotonically increasing function having a rate of change which is equal to or greater than 1.

Next, the stress estimating device 10 extracts a plurality of types of feature value from the biometric data which is the state data in the learning data, and generates feature value data. Then, the stress estimating device 10 associates mapped correct answer data with each piece of feature value data, and generates a dataset of the feature value data and the mapped correct answer data.

Furthermore, the stress estimating device 10 selects types of feature value data to be used for machine learning as learning data from the feature value data extracted from the state data (step S4). At this time, the stress estimating device 10 calculates a correlation value representing a degree of correlation between each type of feature value data and correct answer data, and selects a type of feature value data on the basis of the correlation values.

Then, the stress estimating device 10 performs machine learning using, as learning data, datasets of feature value data selected as mentioned above, and correct answer data mapped onto the nonlinear space, and generates a stress estimation model (step S5). Specifically, the stress estimating device 10 performs the machine learning using, as learning data, all datasets including the selected types of feature value data, using the feature value data as an explanatory variable, and using the mapped correct answer data as a response variable. In this manner, the stress estimating device 10 generates a stress estimation model that calculates, as a value mapped onto the nonlinear space similarly to a PSS score mentioned above, a stress value including a value from 0 to 40 using biometric data about a person U as an input.

Next, an operation of estimating stress on a target person using the generated stress calculation model is explained with reference to the flowchart in FIG. 5. Note that it is assumed that a scene where stress on a target person is estimated is a scene where the person is in a predetermined situation such as a situation where she/he is doing her/his duties at the workplace.

First, the stress estimating device 10 acquires state data to be used for estimating stress on the target person U. Specifically, the data acquiring unit 11 obtains, by measurement, and acquires state data which is biometric data from the person U in a case where she/he is in a situation such as a situation where she/he is doing her/his duties at the workplace which is a scene where stress on the target person U is actually estimated (step S11).

Next, the stress estimating device 10 extracts feature value data from the biometric data (step S12). At this time, the stress estimating device 10 extracts feature value data of the same types as types of the feature value data selected as learning data when the model is generated as mentioned above.

Then, by inputting the extracted feature value data to the stress estimation model, the stress estimating device 10 calculates a stress value which is an output therefrom (step S13). Furthermore, the stress estimating device 10 inverse-maps the calculated stress value onto a linear space (step S14). At this time, the stress estimating device 10 inverse-maps, onto the linear space, the stress value calculated using the stress estimation model using an inverse function of a function used when correct answer data is mapped onto the nonlinear space before learning.

Thereafter, the stress estimating device 10 outputs information based on the stress value inverse-mapped onto the linear space (step S15). For example, the stress estimating device 10 outputs stress information about the target person U such as the stress value itself or an alert based on the stress value.

As mentioned above, the stress estimating device 10 mentioned above generates the model by learning after correct answer data which is stress values in learning data is mapped onto the nonlinear space. Thereby, even in a case where data in which stress values are concentrated extremely around the median is collected as learning data, a model with less concentration of stress values around the median can be generated. As a result, it is possible to estimate stress more precisely.

Note that the present invention can be applied not only to estimation of stress on a person as mentioned above, but also to estimation of the physical condition of a person like physical and mental fatigue, or inner condition. In this case, using, as correct answer data mentioned above, data representing physical condition, for example data like scores based on subjective data related to the fatigue or condition of people, it is possible to estimate a value representing the physical condition of a person similarly to the manner mentioned above.

### Second Exemplary Embodiment

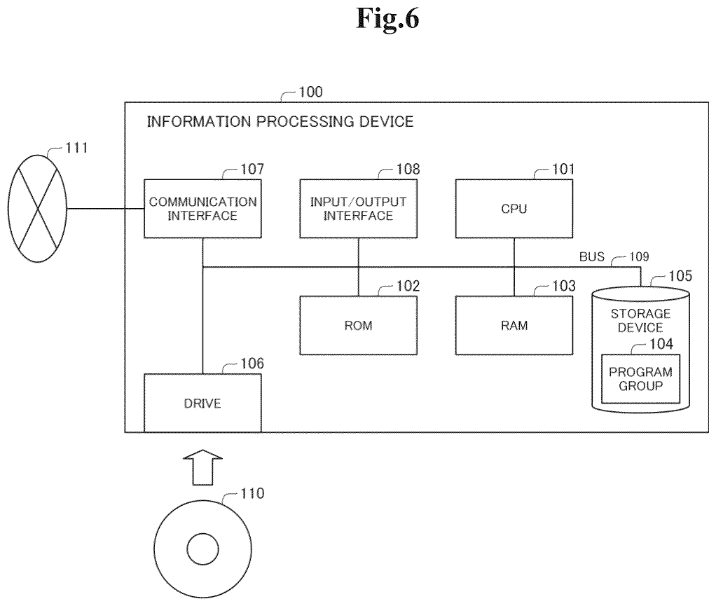

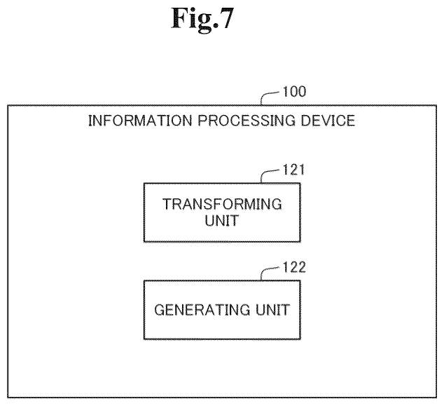

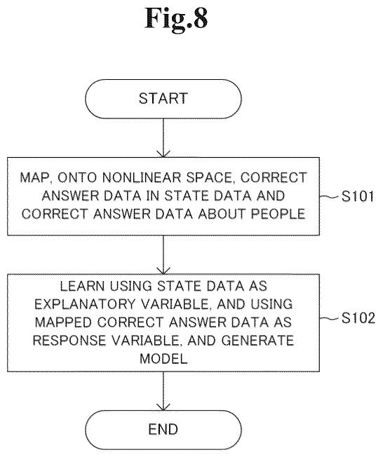

Next, a second exemplary embodiment of the present invention is explained with reference to FIG. 6 to FIG. 8. FIG. 6 to FIG. 7 are block diagrams depicting the configuration of an information processing device in the second exemplary embodiment, and FIG. 8 is a flowchart depicting an operation performed by the information processing device. Note that the present embodiment illustrates outlines of the configurations of the stress estimating device and the stress estimation method explained in the exemplary embodiment mentioned above.

First, the hardware configuration of an information processing device 100 in the present embodiment is explained with reference to FIG. 6. The information processing device 100 is configured using a typical information processing device, and has a hardware configuration as described below as an example.
Central Processing Unit (CPU) 101 (arithmetic device)
Read Only Memory (ROM) 102 (storage device)
Random Access Memory (RAM) 103 (storage device)
Program group 104 to be loaded to RAM 103
Storage device 105 having stored thereon the program group 104
Drive 106 that performs reading and writing on a storage medium 110 outside the information processing device
Communication interface 107 connected to a communication network 111 outside the information processing device
Input/output interface 108 for performing input/output of data
Bus 109 connecting the respective constituent elements

Then, the information processing device 100 can construct and have a transforming unit 121 and a generating unit 122 depicted in FIG. 7 through acquisition of the program group 104 and execution thereof by the CPU 101. Note that the program group 104 is stored on, for example, the storage device 105 or the ROM 102 in advance, is loaded to the RAM 103 by the CPU 101, and is executed by the CPU 101 as needed. In addition, the program group 104 may be supplied to the CPU 101 via the communication network 111, or may be stored on the storage medium 110 in advance, read out by the drive 106, and supplied to the CPU 101. It should be noted that the transforming unit 121 and the generating unit 122 mentioned above may be constructed using electronic circuits dedicated for realizing the means.

Note that FIG. 6 depicts an example of the hardware configuration of the information processing device 100. The hardware configuration of the information processing device is not limited to that mentioned above. For example, the information processing device may be configured using part of the configuration mentioned above, such as without the drive 106.

Then, by functions of the transforming unit 121 and the generating unit 122 constructed by programs as mentioned above, the information processing device 100 executes the information processing method depicted in the flowchart in FIG. 8.

As depicted in FIG. 8, the information processing device 100 executes processes of:
mapping correct answer data included in learning data onto a nonlinear space, the learning data including state data representing states of predetermined people, and the correct answer data representing the physical condition of the predetermined people at the time of acquisition of the state data (step S101); and
generating an estimation model to be used for estimating the physical condition of a target person by learning using the state data as an explanatory variable, and using the mapped correct answer data as a response variable (step S102).

By being configured in the manners described above, the present invention generates the estimation model by learning after mapping, onto the nonlinear space, correct answer data representing physical condition in learning data. Thereby, even in a case where data in which values representing physical condition are extremely concentrated around the median is collected as learning data, it is possible to generate a model with less concentration of the values representing the physical condition around the median, and to estimate physical condition more precisely.

### Supplementary Note

Part of or the whole of the exemplary embodiments described above can also be described as in the following supplementary notes. Hereinbelow, outlines of the configurations of an information processing method, an information processing device, and a program in the present invention are explained. It should be noted that the present invention is not limited to the following configurations.

### (Supplementary Note 1)

An information processing method comprising:
mapping correct answer data included in learning data onto a nonlinear space, the learning data including state data representing a state of a predetermined person, and the correct answer data representing physical condition of the predetermined person at time of acquisition of the state data; and
generating an estimation model to be used for estimating physical condition of a target person by learning using the state data as an explanatory variable, and the mapped correct answer data as a response variable.

### (Supplementary Note 2)

The information processing method according to supplementary note 1, comprising:
calculating a value representing physical condition by inputting, to the estimation model, target person state data representing a state of the target person acquired from the target person; and
inverse-mapping the calculated value representing the physical condition onto a linear space.

### (Supplementary Note 3)

The information processing method according to supplementary note 1 or 2, wherein the correct answer data is mapped onto the nonlinear space using a monotonically increasing function having a rate of change which is equal to or greater than 1.

### (Supplementary Note 4)

The information processing method according to any one of supplementary notes 1 to 3, wherein the correct answer data is mapped onto the nonlinear space using a function for which an inverse function can be defined.

### (Supplementary Note 5)

The information processing method according to any one of supplementary notes 1 to 4, wherein
the state data includes a plurality of pieces of feature value data extracted from measurement data obtained by measurement from the predetermined person, and
the estimation model is generated by learning using, as an explanatory variable, a piece of the feature value data that is selected from a plurality of pieces of the feature value data on a basis of the correct answer data.

### (Supplementary Note 6)

The information processing method according to supplementary note 5, wherein the estimation model is generated by calculating a degree of correlation between each type of the feature value data and the correct answer data, and learning using, as an explanatory variable, a piece of the feature value data that is selected on a basis of the calculated degree of correlation.

### (Supplementary Note 7)

The information processing method according to supplementary note 5 or 6, comprising:
calculating a value representing physical condition by inputting, to the estimation model, target person feature value data which is of the same type as the selected piece of the feature value data, and extracted from target person measurement data obtained by measurement from the target person; and
inverse-mapping the calculated value representing the physical condition onto a linear space.

### (Supplementary Note 8)

An information processing device comprising:
a transforming unit that maps correct answer data included in learning data onto a nonlinear space, the learning data including state data representing a state of a predetermined person, and the correct answer data representing physical condition of the predetermined person at time of acquisition of the state data; and
a generating unit that generates an estimation model to be used for estimating physical condition of a target person by learning using the state data as an explanatory variable, and the mapped correct answer data as a response variable.

### (Supplementary Note 9)

The information processing device according to supplementary note 8, comprising:
a calculating unit that calculates a value representing physical condition by inputting, to the estimation model, target person state data representing a state of the target person acquired from the target person; and
an inverse transforming unit that inverse-maps the calculated value representing the physical condition onto a linear space.

### (Supplementary Note 10)

The information processing device according to supplementary note 8 or 9, wherein the transforming unit maps the correct answer data onto the nonlinear space using a monotonically increasing function having a rate of change which is equal to or greater than 1.

### (Supplementary Note 11)

The information processing device according to any one of supplementary notes 8 to 10, wherein the transforming unit maps the correct answer data onto the nonlinear space using a function for which an inverse function can be defined.

### (Supplementary Note 12)

The information processing device according to any one of supplementary notes 8 to 11, wherein
the state data includes a plurality of pieces of feature value data extracted from measurement data obtained by measurement from the predetermined person, and
the generating unit generates the estimation model by learning using, as an explanatory variable, a piece of the feature value data that is selected from a plurality of pieces of the feature value data on a basis of the correct answer data.

### (Supplementary Note 13)

The information processing device according to supplementary note 12, wherein the generating unit generates the estimation model by calculating a degree of correlation between each type of the feature value data and the correct answer data, and learning using, as an explanatory variable, a piece of the feature value data that is selected on a basis of the calculated degree of correlation.

### (Supplementary Note 14)

The information processing device according to supplementary note 12 or 13, comprising:
a calculating unit that calculates a value representing physical condition by inputting, to the estimation model, target person feature value data which is of the same type as the selected piece of the feature value data, and extracted from target person measurement data obtained by measurement from the target person; and
an inverse transforming unit that inverse-maps the calculated value representing the physical condition onto a linear space.

### (Supplementary Note 15)

A computer readable storage medium having stored thereon a program for causing an information processing device to execute processes of:
mapping correct answer data included in learning data onto a nonlinear space, the learning data including state data representing a state of a predetermined person, and the correct answer data representing physical condition of the predetermined person at time of acquisition of the state data; and
generating a physical condition estimation model to be used for estimating physical condition of a target person by learning using the state data as an explanatory variable, and the mapped correct answer data as a response variable.

### (Supplementary Note 16)

The computer readable storage medium having stored thereon the program according to supplementary note 15 for causing the information processing device to execute processes of:
calculating a value representing physical condition by inputting, to the estimation model, target person state data representing a state of the target person acquired from the target person; and
inverse-mapping the calculated value representing the physical condition onto a linear space.

### REFERENCE SIGNS LIST

10 stress estimating device
11 data acquiring unit
12 preprocessing unit
13 learning unit
14 calculating unit
15 output unit
16 person information storage unit
17 model storage unit
20 input device
30 display device
100 information processing device
101 CPU
102 ROM
103 RAM
104 program group
105 storage device
106 drive
107 communication interface
108 input/output interface
109 bus
110 storage medium
111 communication network
121 transforming unit
122 generating unit

What is claimed is:

1. An information processing method comprising:
mapping correct answer data included in learning data onto a nonlinear space, the learning data including state data representing a state of a predetermined person, and the correct answer data representing physical condition of the predetermined person at time of acquisition of the state data; and
generating an estimation model to be used for estimating physical condition of a target person by learning using the state data as an explanatory variable, and the mapped correct answer data as a response variable.

2. The information processing method according to claim 1, comprising:
calculating a value representing physical condition by inputting, to the estimation model, target person state data representing a state of the target person acquired from the target person; and
inverse-mapping the calculated value representing the physical condition onto a linear space.

3. The information processing method according to claim 1, wherein the correct answer data is mapped onto the nonlinear space using a monotonically increasing function having a rate of change which is equal to or greater than 1.

4. The information processing method according to claim 1, wherein the correct answer data is mapped onto the nonlinear space using a function for which an inverse function can be defined.

5. The information processing method according to claim 1, wherein
the state data includes a plurality of pieces of feature value data extracted from measurement data obtained by measurement from the predetermined person, and
the estimation model is generated by learning using, as an explanatory variable, a piece of the feature value data that is selected from a plurality of pieces of the feature value data on a basis of the correct answer data.

6. The information processing method according to claim 5, wherein the estimation model is generated by calculating a degree of correlation between each type of the feature value data and the correct answer data, and learning using, as an explanatory variable, a piece of the feature value data that is selected on a basis of the calculated degree of correlation.

7. The information processing method according to claim 5, comprising:
calculating a value representing physical condition by inputting, to the estimation model, target person feature value data which is of the same type as the selected piece of the feature value data, and extracted from target person measurement data obtained by measurement from the target person; and
inverse-mapping the calculated value representing the physical condition onto a linear space.

8. An information processing device comprising:
at least one memory configured to store instructions; and
at least one processor configured to execute instructions to:
map correct answer data included in learning data onto a nonlinear space, the learning data including state data representing a state of a predetermined person, and the correct answer data representing physical condition of the predetermined person at time of acquisition of the state data; and
generate an estimation model to be used for estimating physical condition of a target person by learning using the state data as an explanatory variable, and the mapped correct answer data as a response variable.

9. The information processing device according to claim 8, wherein
the at least one processor is configured to execute the instructions to:
calculate a value representing physical condition by inputting, to the estimation model, target person state data representing a state of the target person acquired from the target person; and
inverse map the calculated value representing the physical condition onto a linear space.

10. The information processing device according to claim 8, wherein the at least one processor is configured to execute the instructions to map the correct answer data onto the nonlinear space using a monotonically increasing function having a rate of change which is equal to or greater than 1.

11. The information processing device according to claim 8, wherein the at least one processor is configured to execute the instructions to map the correct answer data onto the nonlinear space using a function for which an inverse function can be defined.

12. The information processing device according to claim 8, wherein
the state data includes a plurality of pieces of feature value data extracted from measurement data obtained by measurement from the predetermined person, and
the at least one processor is configured to execute the instructions to generate the estimation model by learning using, as an explanatory variable, a piece of the feature value data that is selected from a plurality of pieces of the feature value data on a basis of the correct answer data.

13. The information processing device according to claim 12, wherein the at least one processor is configured to execute the instructions to generate the estimation model by calculating a degree of correlation between each type of the feature value data and the correct answer data, and learning using, as an explanatory variable, a piece of the feature value data that is selected on a basis of the calculated degree of correlation.

14. The information processing device according to claim 12, wherein
the at least one processor is configured to execute the instructions to:
calculate a value representing physical condition by inputting, to the estimation model, target person feature value data which is of the same type as the selected piece of the feature value data, and extracted from target person measurement data obtained by measurement from the target person; and
inverse map the calculated value representing the physical condition onto a linear space.

15. A non-transitory computer readable storage medium having stored thereon a program comprising instructions for causing an information processing device to execute processes of:
mapping correct answer data included in learning data onto a nonlinear space, the learning data including state data representing a state of a predetermined person, and the correct answer data representing physical condition of the predetermined person at time of acquisition of the state data; and
generating an estimation model to be used for estimating physical condition of a target person by learning using the state data as an explanatory variable, and the mapped correct answer data as a response variable.

16. The non-transitory computer readable storage medium having stored thereon the program according to claim 15, wherein,
the program comprises instructions for causing the information processing device to execute processes of:
calculating a value representing physical condition by inputting, to the estimation model, target person state data representing a state of the target person acquired from the target person; and
inverse-mapping the calculated value representing the physical condition onto a linear space.
